(19) United States
(12) Patent Application Publication
(10) Pub. No.: US 2016/0067255 A1
(43) Pub. Date: Mar. 10, 2016

(54) METHODS OF TREATING OR PREVENTING HIV IN PATIENTS USING A COMBINATION OF TENOFOVIR ALAFENAMIDE AND DOLUTEGRAVIR

(71) Applicant: Gilead Sciences, Inc., Foster City, CA (US)

(72) Inventors: Darius M. Babusis, Sunnyvale, CA (US); Adrian S. Ray, Burlingame, CA (US); Jianhong Wang, Foster City, CA (US)

(21) Appl. No.: 14/842,609

(22) Filed: Sep. 1, 2015

Related U.S. Application Data
(60) Provisional application No. 62/045,972, filed on Sep. 4, 2014.

(51) Int. Cl. A61K 31/5365; A61K 31/506; A61K 31/675

(52) U.S. Cl. CPC A61K 31/5365; A61K 31/675; A61K 31/506

(57) ABSTRACT

The disclosure describes methods for treating or preventing HIV in a patient using a combination of tenofovir alafenamide and dolutegravir, and to compositions containing such compounds.

### CROSS REFERENCE TO RELATED APPLICATIONS

This non-provisional application claims the benefit of Provisional Application No. 62/045,972, filed Sep. 4, 2014, which is incorporated herein by reference.

### FIELD OF THE INVENTION

Methods for treating or preventing HIV in a patient using a combination of tenofovir alafenamide and dolutegravir are disclosed, and compositions containing such compounds.

### BACKGROUND OF THE INVENTION

Tenofovir alafenamide (TAF) is a nucleotide reverse transcriptase inhibitor and is currently under development for the treatment and prevention of HIV. TAF has greater antiviral activity and a lower incidence of adverse side effects when compared to tenofovir disoproxil.

Dolutegravir is an integrase inhibitor approved for the treatment of HIV infection.

Although different combinations of antiretroviral drugs have been developed for the treatment of HIV, a need still exists for alternative HIV treatment regimens.

### SUMMARY OF THE INVENTION

One embodiment provides a method for treating or preventing HIV in a patient comprising administering to the patient an effective amount of a compound of Formula I:

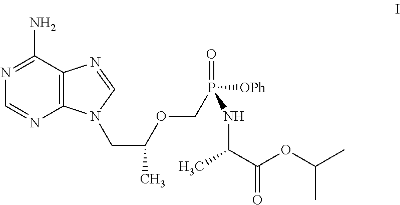 [SMILES: CC(C)OC(=O)[C@H](C)N[P@](=O)(CO[C@H](C)Cn1cnc2c(N)ncnc21)Oc1ccccc1]

or a pharmaceutical composition thereof; and
an effective amount of a compound of formula II:

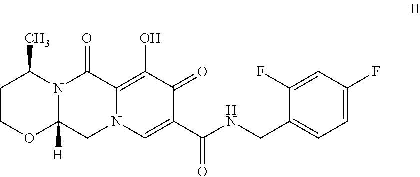 [SMILES: C[C@@H]1CCO[C@H]2Cn3cc(C(=O)NCc4ccc(F)cc4F)c(=O)c(O)c3C(=O)N12]

or a pharmaceutical composition thereof.

In another embodiment, the exposure of the compound of formula I in the patient is increased when coadministered with the compound of formula II, relative to the exposure of the compound of formula I when dosed in the absence of the compound of formula II.

In another embodiment, the Cₘₐₓ, of the compound of formula I in the patient is greater when coadministered with the compound of formula II, relative to the Cₘₐₓ of the compound of formula I when dosed in the absence of the compound of formula II.

In another embodiment, the AUC of the compound of formula I in the patient is greater when coadministered with the compound of formula II, relative to the AUC of the compound of formula I when dosed in the absence of the compound of formula II.

In another embodiment, the compound of formula I is in a pharmaceutical composition comprising the compound of formula I and fumarate.

In another embodiment, the compound of formula I is in a pharmaceutical composition comprising the compound of formula I and fumaric acid.

In another embodiment, the compound of formula II is in a pharmaceutical composition comprising the compound of formula II and sodium.

In another embodiment, the compound of formula I and the compound of formula II are coadministered in separate dosage forms.

In another embodiment, the compound of formula I and the compound of formula II are coadministered in a single dosage form.

In another embodiment, the compound of formula I and the compound of formula II are orally coadministered.

Another embodiment provides a method for increasing the bioavailability of the compound of Formula I:

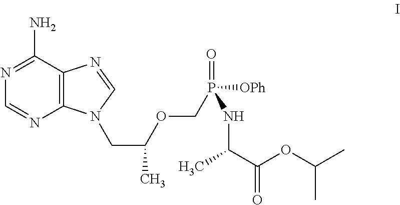 [SMILES: CC(C)OC(=O)[C@H](C)N[P@](=O)(CO[C@H](C)Cn1cnc2c(N)ncnc21)Oc1ccccc1]

comprising administering to a patient an effective amount of the compound of formula I or a pharmaceutical composition thereof; and administering to the patient an effective amount of a compound of formula II:

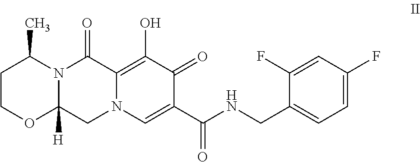 [SMILES: C[C@@H]1CCO[C@H]2Cn3cc(C(=O)NCc4ccc(F)cc4F)c(=O)c(O)c3C(=O)N12]

or a pharmaceutical composition thereof; wherein the exposure of the compound of formula I in the patient is increased compared to administration of the compound of formula I in the absence of the compound of formula II.

In another embodiment, compound II increases the bioavailability of compound I in a patient infected by HIV.

In another embodiment, compound II increases the oral bioavailability of compound I in a patient infected by HIV.

In another embodiment, compound II increases the bioavailability of compound I by inhibiting compound I metabolism.

In another embodiment, compound II increases the bioavailability of compound I by inhibiting compound I intestinal efflux.

In another embodiment, compound II increases the bioavailability of compound I by inhibiting Pgp (P-glycoprotein, also referred to as multidrug resistance protein 1 (MDR1) or ABCB1) activity.

In another embodiment, compound II increases the bioavailability of compound I by inhibiting BCRP (Breast Cancer Resistance Protein, also referred to as ABCG2) activity.

Another embodiment provides a pharmaceutically acceptable composition comprising:

a compound of formula I:

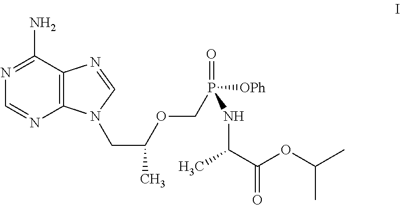 [SMILES: CC(C)OC(=O)[C@H](C)N[P@](=O)(CO[C@H](C)Cn1cnc2c(N)ncnc21)Oc1ccccc1]

and a compound of formula II:

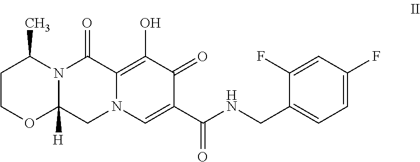 [SMILES: C[C@@H]1CCO[C@H]2Cn3cc(C(=O)NCc4ccc(F)cc4F)c(=O)c(O)c3C(=O)N12]

and a pharmaceutically acceptable carrier.

Another embodiment provides further administration of at least one antiretroviral compound.

In certain embodiments, a method for treating or preventing an HIV infection in a human having or at risk of having the infection is provided, comprising administering to the human a therapeutically effective amount of dolutegravir and tenofovir alafenamide, or a pharmaceutical composition thereof, in combination with a therapeutically effective amount of one or more (e.g., one, two, three, one or two, or one to three) additional therapeutic agents. In one embodiment, a method for treating an HIV infection in a human having or at risk of having the infection is provided, comprising administering to the human a therapeutically effective amount of dolutegravir and tenofovir alafenamide, or a pharmaceutical composition thereof, in combination with a therapeutically effective amount of one or more (e.g., one, two, three, one or two, or one to three) additional therapeutic agents.

In certain embodiments, the present disclosure provides a method for treating an HIV infection, comprising administering to a patient in need thereof a therapeutically effective amount of dolutegravir and tenofovir alafenamide, or a pharmaceutically acceptable composition thereof, in combination with a therapeutically effective amount of one or more additional therapeutic agents which are suitable for treating an HIV infection.

In one embodiment, pharmaceutical compositions comprising dolutegravir and tenofovir alafenamide, in combination with one or more (e.g., one, two, three, one or two, or one to three) additional therapeutic agents, and a pharmaceutically acceptable carrier, diluent or excipient are provided.

In one embodiment, kits comprising dolutegravir and tenofovir alafenamide, or a pharmaceutical composition thereof, in combination with one or more (e.g., one, two, three, one or two, or one to three) additional therapeutic agents are provided.

In the above embodiments, the additional therapeutic agent may be an anti-HIV agent. For example, in some embodiments, the additional therapeutic agent is selected from the group consisting of HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors (e.g., CCR5 inhibitors, gp41 inhibitors (i.e., fusion inhibitors) and CD4 attachment inhibitors), CXCR4 inhibitors, gp120 inhibitors, G6PD and NADH-oxidase inhibitors, HIV vaccines, HIV maturation inhibitors, latency reversing agents (e.g., histone deacetylase inhibitors, proteasome inhibitors, protein kinase C (PKC) activators, and BRD4 inhibitors), compounds that target the HIV capsid (“capsid inhibitors”; e.g., capsid polymerization inhibitors or capsid disrupting compounds, HIV nucleocapsid p7 (NCp7) inhibitors, HIV p24 capsid protein inhibitors), pharmacokinetic enhancers, immune-based therapies (e.g., Pd-1 modulators, Pd-L1 modulators, CTLA4 modulators, toll like receptors modulators, IL-15 agonists, HIV antibodies, bispecific antibodies and “antibody-like” therapeutic proteins (e.g., DARTs®, Duobodies®, Bites®, XmAbs®, TandAbs®, Fab derivatives) including those targeting HIV gp120 or gp41, combination drugs for HIV, HIV p17 matrix protein inhibitors, IL-13 antagonists, Peptidyl-prolyl cis-trans isomerase A modulators, Protein disulfide isomerase inhibitors, Complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, Integrin antagonists, Nucleoprotein inhibitors, Splicing factor modulators, COMM domain containing protein 1 modulators, HIV Ribonuclease H inhibitors, Retrocyclin modulators, CDK-9 inhibitors, Dendritic ICAM-3 grabbing nonintegrin 1 inhibitors, HIV GAG protein inhibitors, HIV POL protein inhibitors, Complement Factor H modulators, Ubiquitin ligase inhibitors, Deoxycytidine kinase inhibitors, Cyclin dependent kinase inhibitors Proprotein convertase PC9 stimulators, ATP dependent RNA helicase DDX3X inhibitors, reverse transcriptase priming complex inhibitors, HIV gene therapy, PI3K inhibitors, compounds such as those disclosed in WO 2013/006738 (Gilead Sciences), US 2013/0165489 (University of Pennsylvania), WO 2013/091096A1 (Boehringer Ingelheim), WO 2009/062285 (Boehringer Ingelheim), US20140221380 (Japan Tobacco), US20140221378 (Japan Tobacco), WO 2010/130034 (Boehringer Ingelheim), WO 2013/159064 (Gilead Sciences), WO 2012/145728 (Gilead Sciences), WO2012/003497 (Gilead Sciences), WO2014/100323 (Gilead Sciences), WO2012/145728 (Gilead Sciences), WO2013/159064 (Gilead Sciences) and WO 2012/003498 (Gilead Sciences) and WO 2013/006792 (Pharma Resources), and other drugs for treating HIV, and combinations thereof.

In certain embodiments, the additional therapeutic is selected from the group consisting of HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof.

In certain embodiments, tenofovir alafenamide and dolutegravir are formulated as a tablet, which may optionally contain one or more other compounds useful for treating HIV. In certain embodiments, the tablet can contain another active ingredient for treating HIV, such as HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof. In certain embodiments, such tablets are suitable for once daily dosing.

In certain embodiments, the additional therapeutic agent is selected from one or more of:

(1) Combination drugs selected from the group consisting of ATRIPLA® (efavirenz+tenofovir disoproxil fumarate+emtricitabine), COMPLERA® (EVIPLERA®, rilpivirine+tenofovir disoproxil fumarate+emtricitabine), STRIBILD® (elvitegravir+cobicistat+tenofovir disoproxil fumarate+emtricitabine), lamivudine+nevirapine+zidovudine, atazanavir sulfate+cobicistat, darunavir+cobicistat, efavirenz+lamivudine+tenofovir disoproxil fumarate, Vacc-4x+romidepsin, APH-0812, raltegravir+lamivudine, KALETRA® (ALUVIA®, lopinavir+ritonavir), atazanavir sulfate+ritonavir, COMBIVIR® (zidovudine+lamivudine, AZT+3TC), EPZICOM® (Livexa®, abacavir sulfate+lamivudine, ABC+3TC), TRIZIVIR® (abacavir sulfate+zidovudine+lamivudine, ABC+AZT+3TC), TRUVADA® (tenofovir disoproxil fumarate+emtricitabine, TDF+FTC), tenofovir+lamivudine, atazanavir+cobicistat, doravirine+lamivudine+tenofovir disoproxil fumarate, doravirine+lamivudine+tenofovir disoproxil and lamivudine+tenofovir disoproxil fumarate;

(2) HIV protease inhibitors selected from the group consisting of amprenavir, atazanavir, fosamprenavir, fosamprenavir calcium, indinavir, indinavir sulfate, lopinavir, ritonavir, nelfinavir, nelfinavir mesylate, saquinavir, saquinavir mesylate, tipranavir, brecanavir, darunavir, DG-17, TMB-657 (PPL-100), TMC-310911, and TMB-657;

(3) HIV non-nucleoside or non-nucleotide inhibitors of reverse transcriptase selected from the group consisting of delavirdine, delavirdine mesylate, nevirapine, (+), etravirine, dapivirine, doravirine, rilpivirine, efavirenz, KM023, VM-1500, lentinan, AIC-292 and KM-023;

(4) HIV nucleoside or nucleotide inhibitors of reverse transcriptase selected from the group consisting of VIDEX® and VIDEX® EC (didanosine, ddl), zidovudine, emtricitabine, didanosine, stavudine, zalcitabine, lamivudine, censavudine, abacavir, abacavir sulfate, amdoxovir, elvucitabine, alovudine, phosphazid, fozivudine tidoxil, apricitabine, amdoxovir, KP-1461, fosalvudine tidoxil, tenofovir, tenofovir disoproxil, tenofovir disoproxil fumarate, tenofovir disoproxil hemifumarate, adefovir, adefovir dipivoxil, and festinavir;

(5) HIV integrase inhibitors selected from the group consisting of curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, elvitegravir and cabotegravir;

(6) HIV non-catalytic site, or allosteric, integrase inhibitors (NCINI) selected from the group consisting of CX-05168, CX-05045 and CX-14442;

(7) HIV gp41 inhibitors selected from the group consisting of enfuvirtide, sifuvirtide and albuvirtide;

(8) HIV entry inhibitors selected from the group consisting of cenicriviroc;

(9) HIV gp120 inhibitors selected from the group consisting of Radha-108 (Receptol) and BMS-663068;

(10) CCR5 inhibitors selected from the group consisting of aplaviroc, vicriviroc, maraviroc, cenicriviroc, PRO-140, Adaptavir (RAP-101), nifeviroc (TD-0232), TD-0680, TBR-220 (TAK-220) and vMIP (Haimipu);

(11) CD4 attachment inhibitors selected from the group consisting of ibalizumab;

(12) CXCR4 inhibitors selected from the group consisting of plerixafor, ALT-1188, vMIP and Haimipu;

(13) Pharmacokinetic enhancers selected from the group consisting of cobicistat and ritonavir;

(14) Immune-based therapies selected from the group consisting of dermaVir, interleukin-7, lexgenleucel-T (VRX-496), plaquenil (hydroxychloroquine), proleukin (aldesleukin, IL-2), interferon alfa, interferon alfa-2b, interferon alfa-n3, pegylated interferon alfa, interferon gamma, hydroxyurea, mycophenolate mofetil (MPA) and its ester derivative mycophenolate mofetil (MMF), WF-10, ribavirin, IL-2, IL-2 XL, IL-12, polymer polyethyleneimine (PEI), Gepon, VGV-1, MOR-22, toll-like receptors modulators (tlr1, tlr2, tlr3, tlr4, tlr5, tlr6, tlr7, tlr8, tlr9, tlr10, tlr11, tlr12 and tlr13), BMS-936559, rintatolimod and IR-103;

(15) HIV vaccines selected from the group consisting of peptide vaccines, recombinant subunit protein vaccines, live vector vaccines, DNA vaccines, virus-like particle vaccines (pseudovirion vaccine), CD4-derived peptide vaccines, vaccine combinations, rgp120 (AIDSVAX), ALVAC HIV (vCP1521)/AIDSVAX B/E (gp120) (RV144), Remune, ITV-1, Contre Vir, Ad5-ENVA-48, DCVax-001 (CDX-2401), PEP-6409, Vacc-4x, Vacc-C5, VAC-3S, multiclade DNA recombinant adenovirus-5 (rAdS), Pennvax-G, VRC-HIV MAB060-00-AB, AVX-101, Tat Oyi vaccine, AVX-201, HIV-LAMP-vax, Ad35, Ad35-GRIN, NAcGM3/VSSP ISA-51, poly-ICLC adjuvanted vaccines, TatImmune, GTU-multiHIV (FIT-06), AGS-004, gp140[delta]V2.TVI+MF-59, rVSVIN HIV-1 gag vaccine, SeV-Gag vaccine, AT-20, DNK-4, Ad35-GRIN/ENV, TBC-M4, HIVAX, HIVAX-2, NYVAC-HIV-PT1, NYVAC-HIV-PT4, DNA-HIV-PT123, Vichrepol, rAAV1-PG9DP, GOVX-B11, GOVX-B21, ThV-01, TUTI-16, VGX-3300, TVI-HIV-1, Ad-4 (Ad4-env Clade C+Ad4-mGag), EN41-FPA2, PreVaxTat, TL-01, SAV-001, AE-H, MYM-V101, CombiHIVvac, ADVAX, MYM-V201, monomeric gp120 HIV-1 subtype C vaccine (Novartis), MVA-CMDR, MVATG-17401, ETV-01, CDX-1401, rcAd26.MOS1.HIV-Env, and DNA-Ad5 gag/pol/nef/nev (HVTN505);

(16) HIV antibodies, bispecific antibodies and “antibody-like” therapeutic proteins (such as DARTs®, Duobodies®, Bites®, XmAbs®, TandAbs®, Fab derivatives) including BMS-936559, TMB-360 and those targeting HIV gp120 or gp41 selected from the group consisting of bavituximab, UB-421, C2F5, C2G12, C4E10, C2F5+C2G12+C4E10, 3-BNC-117, KD-247, PGT145, PGT121, MDXO10 (ipilimumab), VRCO1, A32, 7B2, 10E8, VRC-07-523 and VRC07;

(17) latency reversing agents selected from the group consisting of Histone deacetylase inhibitors such as Romidepsin, vorinostat, panobinostat; Proteasome inhibitors such as Velcade; protein kinase C (PKC) activators such as Indolactam, Prostratin, Ingenol B and DAG-lactones, lonomycin, GSK-343, PMA, SAHA, BRD4 inhibitors, IL-15, JQ1, amphotericin B and disulfram;

(18) HIV nucleocapsid p7 (NCp7) inhibitors selected from the group consisting of azodicarbonamide;

(19) HIV maturation inhibitors selected from the group consisting of BMS-955176 and GSK-2838232;

(20) P13K inhibitors selected from the group consisting of idelalisib, AZD-8186, buparlisib, CLR-457, pictilisib, neratinib, rigosertib, rigosertib sodium, EN-3342, TGR-1202, alpelisib, duvelisib, UCB-5857, taselisib, XL-765, gedatolisib, VS-5584, copanlisib, CAI orotate, perifosine, RG-7666, GSK-2636771, DS-7423, panulisib, GSK-2269557, GSK-2126458, CUDC-907, PQR-309, INCB-040093, pilaralisib, BAY-1082439, puquitinib mesylate, SAR-245409, AMG-319, RP-6530, ZSTK-474, MLN-1117, SF-1126, RV-1729, sonolisib, LY-3023414, SAR-260301 and CLR-1401;

(21) the compounds disclosed in WO 2004/096286 (Gilead Sciences), WO 2006/110157 (Gilead Sciences), WO 2006/015261 (Gilead Sciences), WO 2013/006738 (Gilead Sciences), US 2013/0165489 (University of Pennsylvania), US20140221380 (Japan Tobacco), US20140221378 (Japan Tobacco), WO 2013/006792 (Pharma Resources), WO 2009/062285 (Boehringer Ingelheim), WO 2010/130034 (Boehringer Ingelheim), WO 2013/091096A1 (Boehringer Ingelheim), WO 2013/159064 (Gilead Sciences), WO 2012/145728 (Gilead Sciences), WO2012/003497 (Gilead Sciences), WO2014/100323 (Gilead Sciences), WO2012/145728 (Gilead Sciences), WO2013/159064 (Gilead Sciences) and WO 2012/003498 (Gilead Sciences); and

(22) other drugs for treating HIV selected from the group consisting of REP 9, Cytolin, CYT-107, alisporivir, BanLec, MK-8507, AG-1105, TR-452, MK-8591, REP 9, NOV-205, IND-02, metenkefalin, PGN-007, Acemannan, Gamimune, SCY-635, Prolastin, 1,5-dicaffeoylquinic acid, BIT-225, RPI-MN, VSSP, Hlviral, IMO-3100, SB-728-T, RPI-MN, VIR-576, HGTV-43, MK-1376, rHIV7-shl-TAR-CCR5RZ, MazF gene therapy, BlockAide, ABX-464, SCY-635, naltrexone, AAV-eCD4-Ig gene therapy, TEV-90110, TEV-90112, deferiprone, and PA-1050040 (PA-040).

In certain embodiments, dolutegravir and tenofovir alafenamide, or a pharmaceutical composition thereof, is combined with one, two, three, four or more additional therapeutic agents. In certain embodiments, dolutegravir and tenofovir alafenamide, or a pharmaceutical composition thereof, is combined with two additional therapeutic agents. In other embodiments, dolutegravir and tenofovir alafenamide, or a pharmaceutical composition thereof, is combined with three additional therapeutic agents. In further embodiments, dolutegravir and tenofovir alafenamide, or a pharmaceutical composition thereof, is combined with four additional therapeutic agents. The one, two, three, four or more additional therapeutic agents can be different therapeutic agents selected from the same class of therapeutic agents, and/or they can be selected from different classes of therapeutic agents.

In a particular embodiment, dolutegravir and tenofovir alafenamide, or a pharmaceutical composition thereof is combined with one, two, three, four or more additional therapeutic agents selected from raltegravir, Truvada® (tenofovir disoproxil fumarate+emtricitabine, TDF+FTC), maraviroc, enfuvirtide, Epzicom® (Livexa®, abacavir sulfate+lamivudine, ABC+3TC), Trizivir® (abacavir sulfate+zidovudine+lamivudine, ABC+AZT+3TC), adefovir, adefovir dipivoxil, Stribild® (elvitegravir+cobicistat+tenofovir disoproxil fumarate+emtricitabine), rilpivirine, rilpivirine hydrochloride, raltegravir+lamivudine, Complera® (Eviplera®, rilpivirine+tenofovir disoproxil fumarate+emtricitabine), Cobicistat, Atripla® (efavirenz+tenofovir disoproxil fumarate+emtricitabine), atazanavir sulfate+cobicistat, atazanavir+cobicistat, darunavir+cobicistat, atazanavir, atazanavir sulfate, elvitegravir, Aluvia® (Kaletra®, lopinavir+ritonavir), ritonavir, emtricitabine, atazanavir sulfate+ritonavir, darunavir, lamivudine, Prolastin, fosamprenavir, fosamprenavir calcium, efavirenz, Combivir® (zidovudine+lamivudine, AZT+3TC), etravirine, nelfinavir, nelfininavir mesylate, interferon, didanosine, stavudine, indinavir, indinavir sulfate, tenofovir+lamivudine, zidovudine, nevirapine, saquinavir, saquinavir mesylate, aldesleukin, zalcitabine, tipranavir, amprenavir, delavirdine, delavirdine mesylate, Radha-108 (Receptol), Hiviral, lamivudine+tenofovir disoproxil fumarate, efavirenz+lamivudine+tenofovir disoproxil fumarate, phosphazid, lamivudine+nevirapine+zidovudine, abacavir, abacavir sulfate, tenofovir, tenofovir disoproxil and tenofovir disoproxil fumarate.

In one embodiment, tenofovir alafenamide and dolutegravir are administered with emtricitabine or lamivudine. In one embodiment, tenofovir alafenamide and dolutegravir are administered with emtricitabine. In one embodiment, tenofovir alafenamide and dolutegravir are administered with lamivudine.

In one embodiment, the combination of tenofovir alafenamide and dolutegravir is administered with emtricitabine or lamivudine. In one embodiment, the combination of tenofovir alafenamide and dolutegravir is administered with emtricitabine. In one embodiment, the combination of tenofovir alafenamide and dolutegravir is administered with lamivudine.

In certain embodiments, when dolutegravir and tenofovir alafenamide, or a pharmaceutical composition thereof is combined with one or more additional therapeutic agents as described above, the components of the composition are administered as a simultaneous or sequential regimen. When administered sequentially, the combination may be administered in two or more administrations.

In certain embodiments, dolutegravir and tenofovir alafenamide, or a pharmaceutical composition thereof, is combined with one or more additional therapeutic agents in a unitary dosage form for simultaneous administration to a patient, for example as a solid dosage form for oral administration.

In certain embodiments, dolutegravir and tenofovir alafenamide, or a pharmaceutical composition thereof, is administered with one or more additional therapeutic agents. Co-administration of dolutegravir and tenofovir alafenamide, or a pharmaceutical composition thereof, with one or more additional therapeutic agents generally refers to simultaneous or sequential administration of a compound disclosed herein and one or more additional therapeutic agents, such that therapeutically effective amounts of the compound disclosed herein and one or more additional therapeutic agents are both present in the body of the patient.

Co-administration includes administration of unit dosages of dolutegravir and tenofovir alafenamide, or a pharmaceutical composition thereof, before or after administration of unit dosages of one or more additional therapeutic agents, for example, administration of dolutegravir and tenofovir alafenamide, or a pharmaceutical composition thereof, within seconds, minutes, or hours of the administration of one or more additional therapeutic agents. For example, in some embodiments, a unit dose of dolutegravir and tenofovir alafenamide, or a pharmaceutical composition thereof, is administered first, followed within seconds or minutes by administration of a unit dose of one or more additional therapeutic agents. Alternatively, in other embodiments, a unit dose of one or more additional therapeutic agents is administered first, followed by administration of a unit dose of dolutegravir and tenofovir alafenamide, or a pharmaceutical composition thereof, within seconds or minutes. In some embodiments, a unit dose of dolutegravir and tenofovir alafenamide, or a pharmaceutical composition thereof, is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of dolutegravir and tenofovir alafenamide, or a pharmaceutical composition thereof.

In certain embodiments, dolutegravir and tenofovir alafenamide, or a pharmaceutical composition thereof, are administered orally.

In another embodiment, the combination of tenofovir alafenamide and dolutegravir is administered to the patient once a day.

In another embodiment, the combination of tenofovir alafenamide and dolutegravir is administered to the patient twice a day.

In another embodiment, dolutegravir is administered to the patient at about 25 mg, about 30 mg, about 35 mg, about 40 mg, about 45 mg dose, about 50 mg, about 55 mg, about 60 mg, about 65 mg, about 70 mg, about 75 mg, about 80 mg, about 85 mg, about 90 mg, about 95 mg or about 100 mg, once, twice or three times a day. In another embodiment, dolutegravir is administered to the patient at about 25 mg to 100 mg, at about 25 mg to 75 mg, at about 35 mg to 65 mg or about 45 mg to 55 mg, once or twice per day. In another embodiment, dolutegravir is administered to the patient at about 50 mg, once or twice per day.

In another embodiment, tenofovir alafenamide is administered to the patient at about 1 mg, about 5 mg, about 10 mg, about 15 mg, about 20 mg, about 25 mg dose, about 30 mg, about 35 mg, about 40 mg, about 45 mg or about 50 mg once, twice or three times a day. In another embodiment, tenofovir alafenamide is administered to the patient at about 1 mg to 50 mg or at about 5 mg to 25 mg once per day. In another embodiment, tenofovir alafenamide is administered to the patient at about 5 mg, once per day. In another embodiment, tenofovir alafenamide is administered to the patient at about 10 mg, once per day. In another embodiment, tenofovir alafenamide is administered to the patient at about 25 mg, once per day.

### BRIEF DESCRIPTION OF THE FIGURES

FIG. 1 shows the plasma concentration versus time of TAF Following a 5 mg/kg Oral Administration to Male Beagle Dogs on Day 1 and Day 6.

FIG. 2 shows the plasma Concentration versus time of TFV Following a 5 mg/kg Oral Administration of TAF to Male Beagle Dogs on Day 1 and Day 6.

### DETAILED DESCRIPTION

As described herein, upon co-dosing of dolutegravir with TAF, an increased exposure (AUC) to TAF was observed in dogs. TAF Cₘₐₓ and AUC were higher when co-dosed with dolutegravir compared to TAF being dosed alone. Plasma sampling following oral administration of TAF alone yielded a Cₘₐₓ of 412 nM and an oral exposure of 174 nM·hr. Coadministration of TAF and DTG following 4 daily doses of only DTG yielded a Cₘₐₓ of 818 nM and an oral exposure of 345 nM·hr for TAF. Accordingly, both oral exposure and maximal concentrations (Cₘₐₓ) of TAF in dog increased approximately 2-fold upon coadministration with DTG.

### DEFINITIONS

As used herein, the term “co-administer” refers to administration of two or more agents within a 24 hour period of each other, for example, as part of a clinical treatment regimen. In other embodiments, “co-administer” refers to administration of two or more agents within 2 hours of each other. In other embodiments, “co-administer” refers to administration of two or more agents within 30 minutes of each other. In other embodiments, “co-administer” refers to administration of two or more agents within 15 minutes of each other. In other embodiments, “co-administer” refers to administration at the same time, either as part of a single formulation or as multiple formulations that are administered by the same or different routes.

“Tenofovir alafenamide” or “TAF” is {9-[(R)-2-[[(S)-[[(S)-1-(isopropoxycarbonyl)ethyl]amino]phenoxyphosphinyl]-methoxy]propyl]adenine}:

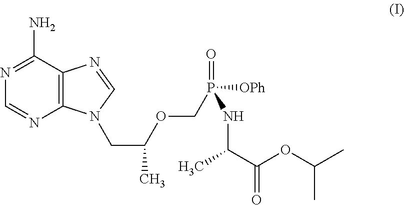 [SMILES: CC(C)OC(=O)[C@H](C)N[P@](=O)(CO[C@H](C)Cn1cnc2c(N)ncnc21)Oc1ccccc1]

TAF may be associated with fumarate, such as monofumarate and hemifumarate.

TFV or Tenofovir is:

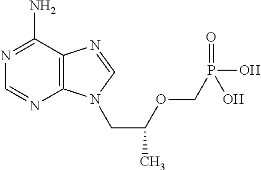 [SMILES: C[C@H](Cn1cnc2c(N)ncnc21)OCP(=O)(O)O]

“Tenofovir disoproxil” or “TD” is 9-[(R)-2-[[bis[[(isopropoxycarbonyl)oxy]methoxy]phosphinyl]methoxy]propyl]adenine. TDF may be associated with fumarate, such as monofumarate.

“Dolutegravir” or DTG is:

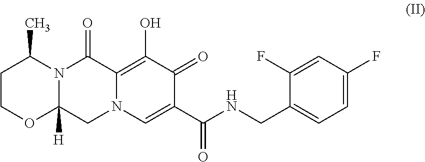 [SMILES: C[C@@H]1CCO[C@H]2Cn3cc(C(=O)NCc4ccc(F)cc4F)c(=O)c(O)c3C(=O)N12]

Dolutegravir may be associated with sodium.

“Emtricitabine” or “FTC” refers to (2R,5S,cis)-4-amino-5-fluoro-1-(2-hydroxymethyl-1,3-oxathiolan-5-yl)-(1H)-pyrimidin-2-one.

Cₘₐₓ is the peak plasma concentration of a drug after administration

Tₘₐₓ is the time at which the Cₘₐₓ, is observed.

AUC or area under the curve is the integral of the concentration-time curve.

AUC_{0-inf} or area under the curve from time 0 to complete elimination of the drug or bioavailability is the fraction of a drug systemically available.

T_{1/2} or elimination half life is the time required for the concentration of the drug to reach half of its original value.

“Therapeutically effective amount” or “effective amount” refers to that amount of the compound being administered which will prevent a condition, or will relieve to some extent one or more of the symptoms of the disorder being treated. Pharmaceutical compositions suitable for use herein include compositions wherein the active ingredients are contained in an amount sufficient to achieve the intended purpose. Determination of a therapeutically effective amount is well within the capability of those skilled in the art, especially in light of the detailed disclosure provided herein. As used herein, treatment refers to inhibition, reduction, elimination or alleviation of a disease as well as prevention.

A method for the treatment or prophylaxis of diseases, disorders, and conditions is provided herein. An example of a disease, disorder, or condition includes, but is not limited to, a retrovirus infection, or a disease, disorder, or condition associated with a retrovirus infection. Retroviruses are RNA viruses and are generally classified into the alpharetrovirus, betaretrovirus, deltaretrovirus, epsilonretrovirus, gammaretrovirus, lentivirus, and spumavirus families. Examples of retroviruses include, but are not limited to, human immunodeficiency virus (HIV).

The active agents may be administered to a human in any conventional manner. While it is possible for the active agents to be administered as compounds, they are preferably administered as a pharmaceutical composition, which can include contact with an acid or base, either in an ionic salt form or in contact with the base or acid (i.e. co-formers) without sharing ions. The salt, acid or base co-former, carrier, or diluent should be acceptable in the sense of being compatible with the other ingredients and not deleterious to the recipient thereof. Examples of carriers or diluents for oral administration include cornstarch, lactose, magnesium stearate, talc, microcrystalline cellulose, stearic acid, povidone, crospovidone, dibasic calcium phosphate, sodium starch glycolate, hydroxypropyl cellulose (e.g., low substituted hydroxypropyl cellulose), hydroxypropylmethyl cellulose (e.g., hydroxypropylmethyl cellulose 2910), sodium lauryl sulfate, mannitol, sodium stearyl fumarate, and talc. Examples of salts and acid or base co-formers include fumarate, hemifumarate, sodium, hydrochloride and the like.

The pharmaceutical compositions may be prepared by any suitable method, such as those methods well known in the art of pharmacy, for example, methods such as those described in Gennaro et al., Remington's Pharmaceutical Sciences (18th ed., Mack Publishing Co., 1990), especially Part 8: Pharmaceutical Preparations and their Manufacture. Such methods include the step of bringing into association the compounds with the carrier or diluent and optionally one or more accessory ingredients. Such accessory ingredients include those conventional in the art, such as, fillers, binders, excipients, disintegrants, lubricants, colorants, flavoring agents, sweeteners, preservatives (e.g., antimicrobial preservatives), suspending agents, thickening agents, emulsifying agents, and/or wetting agents.

In practice, the amount of each compound to be administered ranges from about 0.001 to 100 mg per kg of body weight, such total dose being given at one time or in divided doses. Each compound will be administered as a formulation in association with one or more pharmaceutically acceptable excipients. Alternatively, both compounds will be combined and administered as a formulation in association with one or more pharmaceutically acceptable excipients. The choice of excipient will to a large extent depend on factors such as the particular mode of administration, the effect of the excipient on solubility and stability, and the nature of the dosage form.

Pharmaceutical compositions suitable for the delivery of compounds described herein and methods for their preparation will be readily apparent to those skilled in the art. Such compositions and methods for their preparation may be found, for example, in Remington's Pharmaceutical Sciences, 19th Edition (Mack Publishing Company, 1995).

In the following description of the examples, specific embodiments in which the invention may be practiced are described. These embodiments are described in sufficient detail to enable those skilled in the art to practice the invention. Other embodiments may be utilized and logical and other changes may be made without departing from the scope of the invention. The following detailed description is, therefore, not to be taken in a limiting sense, and the scope of the invention is defined only by the appended claims, along with the full scope of equivalents to which such claims are entitled.

### Examples

### Example 1

### Oral Pharmacokinetics of Tenofovir Alafenamide (TAF) Following Administration Alone or in Combination with Dolutegravir to Male Beagle Dogs

Experimental Design

TAF was dosed at 5 mg/kg (as free base) orally in solution to fasted male beagle dogs (N=6 animals, mean weight 10 kg). On Day 1, TAF was administered alone. On days 2, 3, 4 and 5, 3 mg/kg of Dolutegravir (DTG) was administered alone (as free acid). Both compounds were administered on Day 6. The plasma pharmacokinetic profiles of TAF and its nucleotide metabolite tenofovir (TFV) were determined by LC/MS in samples taken on Day 1 and Day 6 of dosing.

The oral dosing formulation for TAF was 0.1% Hydroxypropylmethylcellulose K100LV, 0.1% Polysorbate 20, 99.8% deionized water. For dolutegravir, the oral dosing formulation was 0.5% Hydroxypropylmethylcellulose LV 100 and 0.1% Tween 20 in deionized water.

The animals were fasted overnight prior to each dose administration and up to 4 hr after dosing. The blood samples were collected on days 1 and 6 predose and at the following time points following dosing: 0.25, 0.5, 1, 2, 4, 6, 8, 12 and 24 hour. The blood samples were collected into Vacutainer™ tubes containing Sodium Fluoride/Potassium Oxalate (BD Biosciences). The blood samples were centrifuged at 4° C. to separate plasma. Plasma samples were frozen and stored in −70° C. freezers.

Plasma was prepared by protein precipitation by adding acetonitrile to a final concentration of 70% in the presence of internal standard. Following filtration to remove precipitated protein, samples were evaporated, reconstituted with mobile phase A (0.2% formate in water) and analyzed using injections of 5 μl for analysis.

Non-fasted dogs had originally been administered a single dose of TAF, but the first study was halted to conform to a fasted animal study design. After a 5-day washout period, low levels (<10 nM) of TFV were still observed in Day 1 pre-dose plasma samples.

Results

Tables 1 to 4 show the plasma pharmacokinetics parameters for TAF and TFV at day 1 and 6.

TABLE 1
Day 1 Mean Plasma Pharmacokinetic Parameters for TAF Following
Oral Administration to Male Beagle Dogs (Mean ± SD, n = 6)

| Parameter | Mean | SD |
|---|---|---|
| AUC₀₋₂₄ₕᵣ (nM · hr) | 174 | 110 |
| Tₘₐₓ (hr) | 0.29 | 0.10 |
| Cₘₐₓ (nM) | 412 | 206 |
| T_{1/2} (hr)^{a} | 0.19 | 0.06 |

^{a}Estimated terminal elimination half-life

TABLE 2
Day 6 Mean Plasma Pharmacokinetic Parameters for
TAF Following Oral Co-Administration with DTG
to Male Beagle Dogs (Mean ± SD, n = 6)

| Parameter | Mean | SD |
|---|---|---|
| AUC₀₋₂₄ₕᵣ (nM · hr) | 345 | 158 |
| Tₘₐₓ (hr) | 0.25 | 0 |
| Cₘₐₓ (nM) | 818 | 396 |
| T_{1/2} (hr)^{a} | 0.21 | 0.03 |

^{a}Estimated terminal elimination half-life

TABLE 3
Day 1 Mean Plasma Pharmacokinetic Parameters for TFV Following
Oral Administration of TAF to Male Beagle Dogs (Mean ± SD, n = 6)

| Parameter | Mean | SD |
|---|---|---|
| AUC₀₋₂₄ₕᵣ (nM · hr) | 1,600 | 300 |
| AUC_{0-inf} (nM · hr) | 2,920 | 1,290 |
| Tₘₐₓ (hr) | 1.25 | 0.61 |
| Cₘₐₓ (nM) | 253 | 49 |
| T_{1/2} (hr)^{a} | 25.0 | 10.6 |

^{a}Estimated terminal elimination half-life

TABLE 4
Day 6 Mean Plasma Pharmacokinetic Parameters for
TFV Following Oral Co-Administration of TAF with
DTG to Male Beagle Dogs (Mean ± SD, n = 6)

| Parameter | Mean | SD |
|---|---|---|
| ALIC₀₋₂₄ₕᵣ (nM · hr) | 1,460 | 180 |
| AUC_{0-inf} (nM · hr) | 2,870 | 550 |
| Tₘₐₓ (hr) | 0.92 | 0.20 |
| Cₘₐₓ (nM) | 203 | 23 |
| T_{1/2} (hr)^{a} | 27.3 | 6.6 |

^{a}Estimated terminal elimination half-life

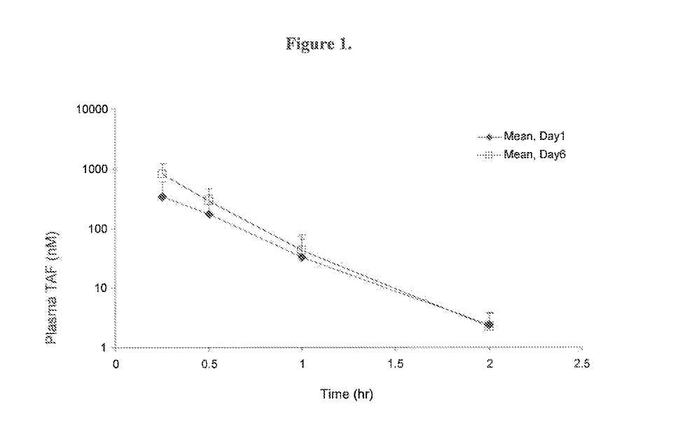

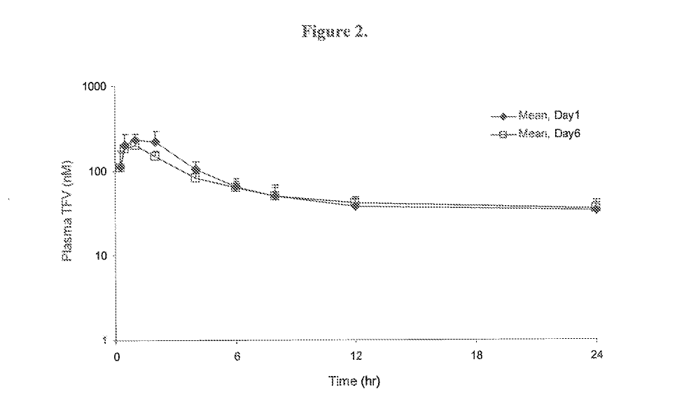

FIG. 1 shows the plasma concentration versus time of TAF Following a 5 mg/kg Oral Administration to Male Beagle Dogs on Day 1 and Day 6 and FIG. 2 shows the plasma Concentration versus time of TFV Following a 5 mg/kg Oral Administration of TAF to Male Beagle Dogs on Day 1 and Day 6.

An increased exposure (AUC) to TAF was observed following DTG administration. TAF Cₘₐₓ and AUC were higher on Day 6 relative to Day 1. Day 1 plasma sampling following oral administration of TAF yielded a Cₘₐₓ of 412 nM and an oral exposure of 174 nM·hr. Coadministration of TAF and DTG following 4 daily doses of DTG yielded a Cₘₐₓ of 818 nM and an oral exposure of 345 nM·hr for TAF. Marginal decreases in mean TFV AUC and Cₘₐₓ were observed on Day 6 versus Day 1.

In conclusion, both oral exposure and maximal concentrations (Cmax) of TAF in dog increased approximately 2-fold upon coadministration with DTG.

We claim:

1. A method for treating or preventing human immunodeficiency virus (HIV) in a patient in need thereof, comprising administering to the patient an effective amount of a compound of Formula I: 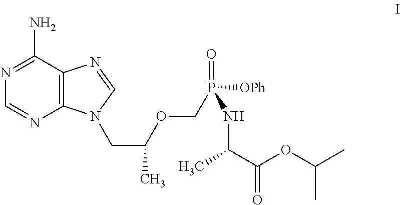 [SMILES: CC(C)OC(=O)[C@H](C)N[P@](=O)(CO[C@H](C)Cn1cnc2c(N)ncnc21)Oc1ccccc1]
or a pharmaceutical composition thereof; and
an effective amount of a compound of Formula II: 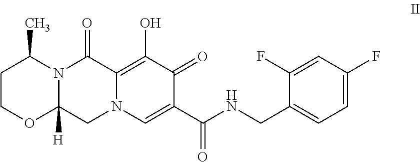 [SMILES: C[C@@H]1CCO[C@H]2Cn3cc(C(=O)NCc4ccc(F)cc4F)c(=O)c(O)c3C(=O)N12]
or a pharmaceutical composition thereof.

2. The method of claim 1, wherein the exposure of the compound of Formula I in the patient is increased when coadministered with the compound of Formula II, relative to the exposure of the compound of Formula I when dosed in the absence of the compound of Formula II.

3. The method of claim 1, wherein the Cₘₐₓ of the compound of formula I in the patient is greater when coadministered with the compound of formula II, relative to the Cₘₐₓ of the compound of formula I when dosed in the absence of the compound of formula II.

4. The method of claim 1, wherein the AUC of the compound of formula I in the patient is greater when coadministered with the compound of formula II, relative to the AUC of the compound of formula I when dosed in the absence of the compound of formula II.

5. The method of claim 1, wherein the compound of formula I is in a pharmaceutical composition comprising fumaric acid and the compound of formula I.

6. The method of claim 1, wherein the compound of formula II is in a pharmaceutical composition comprising the compound of formula II and sodium.

7. The method of claim 1, wherein the compound of formula I and the compound of formula II are coadministered in separate dosage forms.

8. The method of claim 1, wherein the compound of formula I and the compound of formula II are coadministered in a single dosage form.

9. The method of claim 1, further comprising administering emtricitabine.

10. A method for increasing bioavailability of the compound of formula I: 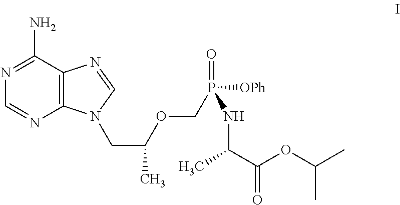 [SMILES: CC(C)OC(=O)[C@H](C)N[P@](=O)(CO[C@H](C)Cn1cnc2c(N)ncnc21)Oc1ccccc1]
comprising administering to a patient an effective amount of the compound of formula I or a pharmaceutical composition thereof; and
administering to the patient an effective amount of a compound of formula II: 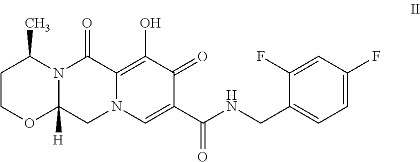 [SMILES: C[C@@H]1CCO[C@H]2Cn3cc(C(=O)NCc4ccc(F)cc4F)c(=O)c(O)c3C(=O)N12]
or a pharmaceutical composition thereof;
wherein the exposure of the compound of formula I in the patient is increased compared to administration of the compound of formula I in the absence of the compound of formula II.

11. The method of claim 10, wherein the patient is infected by human immunodeficiency virus (HIV).

12. The method of claim 10, wherein the compound of formula I is in a pharmaceutical composition comprising the compound of formula I and fumaric acid.

13. The method of claim 10, wherein the compound of formula II is in a pharmaceutical composition comprising the compound of formula II and sodium.

14. The method of claim 10, wherein the compound of formula I and the compound of formula II are coadministered in separate dosage forms.

15. The method of claim 10, wherein the compound of formula I and the compound of formula II are coadministered in a single dosage form.

16. The method of claim 10, wherein the Cₘₐₓ of the compound of formula I is increased relative to the Cₘₐₓ of the compound of formula I administered in the absence of the compound of formula II.

17. The method of claim 10, further comprising administering emtricitabine.

18. A pharmaceutically acceptable composition comprising:
a compound of formula I: 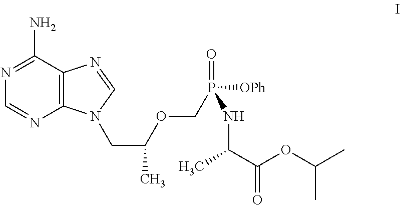 [SMILES: CC(C)OC(=O)[C@H](C)N[P@](=O)(CO[C@H](C)Cn1cnc2c(N)ncnc21)Oc1ccccc1]
and
a compound of formula II: 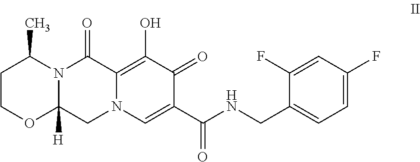 [SMILES: C[C@@H]1CCO[C@H]2Cn3cc(C(=O)NCc4ccc(F)cc4F)c(=O)c(O)c3C(=O)N12]
and a pharmaceutically acceptable carrier.

19. The pharmaceutically acceptable composition of claim 18, wherein the compound of formula I is in contact with fumaric acid and the compound of formula II is in contact with sodium.

20. The pharmaceutically acceptable composition of claim 18, further comprising emtricitabine.

21. A kit comprising:
(1) a composition comprising tenofovir alafenamide and emtricitabine;
(2) a composition comprising dolutegravir;
and instructions for their co-administration.
